(19) United States
(12) Patent Application Publication
(10) Pub. No.: US 2026/0263786 A1
(43) Pub. Date: Sep. 10, 2026

(54) IMPLANTABLE CARDIAC DEVICE

(71) Applicant: Medtronic, Inc., Minneapolis, MN (US)

(72) Inventor: Hassaan M. Khan, Brooklyn Center, MN (US)

(21) Appl. No.: 19/553,519

(22) Filed: Mar. 2, 2026

Related U.S. Application Data
(60) Provisional application No. 63/767,167, filed on Mar. 5, 2025.

(51) Int. Cl. A61N 1/05; A61N 1/368; A61N 1/375; A61N 1/378

(52) U.S. Cl. CPC A61N 1/0573; A61N 1/3684; A61N 1/37512; A61N 1/378

(57) ABSTRACT

An implantable medical device (IMD) comprising: a flexible distal portion comprising: a first electrode extending distally from the flexible distal portion; a flexible proximal portion comprising: a second electrode extending proximally from the flexible proximal portion; and a medial portion extending from the flexible distal portion to the flexible proximal portion, the medial portion defining a rigid housing, wherein the flexible distal portion is configured to be at least partially disposed within a first chamber of a heart of a patient to cause the first electrode to contact wall tissue of the first chamber of the heart, and wherein the flexible proximal portion is configured to be at least partially disposed within a second chamber of the heart to cause the second electrode to contact wall tissue of the second chamber of the heart.

### RELATED APPLICATIONS

This application claims the benefit of U.S. Provisional Application Serial Number 63/767,167, filed March 5, 2025, the entire contents of which are incorporated herein by reference.

### TECHNICAL FIELD

The disclosure relates to medical devices, and more particularly to implantable cardiac devices.

### BACKGROUND

Various types of implantable medical devices (IMDs) have been implanted for treating or monitoring one or more conditions of a patient. Such IMDs may be adapted to monitor or treat conditions or functions relating to heart, muscle, nerve, brain, stomach, endocrine organs or other organs and their related functions. Such IMDs may be associated with leads that position electrodes at a desired location or may be leadless with electrodes integrated with and/or attached to the device housing. These IMDs may have the ability to wirelessly transmit data either to another device implanted in the patient or to another instrument located externally of the patient, or both.

A cardiac device may include an IMD configured to deliver therapy signals to the heart to restore a more normal heart rhythm. Such IMDs sense the electrical activity of the heart, and deliver therapy signals based on the sensed electrical activity, via electrodes. Some IMDs are implanted with a distance from the heart and coupled to one or more leads that intravascularly extend into the heart to position electrodes with respect to cardiac tissue.

### SUMMARY

In general, this disclosure is directed to implantable medical devices (IMDs) configured to sense and deliver electrical signals to tissue of a patient via a plurality of electrodes. More particularly, this disclosure is directed to IMDs configured to deliver pacing signals to cardiac tissue in multiple heart chambers of the patient.

In some examples, a single IMD is wholly implanted within vasculature, e.g., within the heart, of the patient. The IMD may define a flexible distal portion, a flexible proximal portion, and a medial portion connecting the flexible distal portion to the flexible proximal portion. The IMD may include electrode(s) disposed on each of the flexible distal portion and the flexible proximal portion. The distal portion of the IMD may be disposed within one chamber of a heart of the patient and is able to sense signals from and/or deliver therapy signals (e.g., defibrillation signals, pacing signals) to wall tissue of the chamber. The proximal portion may be disposed within another chamber of the heart and may be able to sense signals from and/or deliver therapy signals to wall tissue of the other chamber. The IMD may transmit a defibrillation signal between the defibrillation electrodes to defibrillate cardiac tissue of the patient. The IMD may deliver therapy signals to one or more chambers of the heart sequentially or simultaneously, e.g., to provide single-chamber pacing or dual-chamber pacing to the heart of the patient.

The medial portion may include a rigid body configured to retain electronic components of the IMD. The distal and proximal portions may be flexible and may each define a flexible elongated body. The flexibility of the distal and proximal portions may allow the distal and proximal portions to be implanted at different locations within the heart and/or the vasculature of the patient. The flexible distal and proximal portions may allow for movement of the IMD with the movement of the heart within the patient, e.g., thereby inhibiting unintended detachment of the IMD from the cardiac tissue.

The IMD may sense signals from the patient and determine, based on the sensed signals, whether to deliver one or more of a pacing signal or a defibrillation signal to the patient. The IMD may sense signals from electrode(s) and/or sensor(s) disposed on or within one or more of the distal portion, the medial portion, or the proximal portion. The IMD may determine whether to deliver one or more of the pacing signal or the defibrillation signal to one or more target locations within the heart based on one or more of an electrical stability of one or more chambers of the heart or a hemodynamic status of the patient.

The IMD described herein may provide several advantages over other implantable medical devices (e.g., implantable cardiac devices). The IMD may be entirely disposed within the vasculature of the patient, which may reduce patient discomfort and/or reduce visibility of the IMD. Containing the components of the IMD within the elongated body may reduce a number of leads connecting signal generating components of the IMD (e.g., signal generation circuitry) to signal delivery elements of the IMD (e.g., electrodes, defibrillation electrodes). Disposing the IMD entirely within the heart may further eliminate the need for transvenous routing of leads to the heart. Containing the components of the IMD within the elongated body and disposing the IMD entirely within the vasculature of the patient may eliminate a need for transvenous leads or multiple single-chamber IMDs, which may reduce a complexity of the system while still providing capabilities for sensing signals from and/or delivering electrical signals to multiple chambers of the heart. The design of the IMD may allow the IMD to be implanted at different sites within the heart and/or blood vessels connected to the heart, which may allow for the IMD to be used for a range of different cardiac applications. Implantation of the IMD within the vasculature of the patient may eliminate a need for a subcutaneous pocket for the IMD, which may eliminate a potential source of infection.

In some examples, this disclosure is directed to an implantable medical device (IMD) comprising: a flexible distal portion extending to a device distal end of the IMD, the flexible distal portion comprising: a first electrode extending distally from the device distal end; a flexible proximal portion extending to a device proximal end of the IMD, the flexible proximal portion comprising: a second electrode extending proximally from the device proximal end; and a medial portion extending from the flexible distal portion to the flexible proximal portion, the medial portion defining a rigid housing, wherein the flexible distal portion is configured to be at least partially disposed within a first chamber of a heart of a patient to cause the first electrode to contact wall tissue of the first chamber of the heart, and wherein the flexible proximal portion is configured to be at least partially disposed within a second chamber of the heart to cause the second electrode to contact wall tissue of the second chamber of the heart.

In some examples, this disclosure is directed to a method comprising: advancing an implantable medical device (IMD) within vasculature of a patient into a heart of the patient, the IMD comprising: a flexible distal portion extending to a device distal end of the IMD, the flexible distal portion comprising: a first electrode extending distally from the device distal end, a flexible proximal portion extending to a device proximal end of the IMD, the flexible proximal portion comprising: a second electrode extending proximally from the device proximal end, and a medial portion extending from the flexible distal portion to the flexible proximal portion, the medial portion defining a rigid housing; advancing at least a portion of the flexible distal portion into a first chamber of the heart; affixing the flexible distal portion to wall tissue of the first chamber to cause the first electrode to contact the wall tissue of the first chamber; disposing at least a portion of the flexible proximal portion in a second chamber of the heart; affixing the flexible proximal portion to wall tissue of the second chamber to cause the second electrode to contact the wall tissue of the second chamber; delivering, by the IMD and via the first electrode, a first electrical signal to the wall tissue of the first chamber; and delivering, by the IMD and via the second electrode, a second electrical to the wall tissue of the second chamber.

In some examples, this disclosure is directed to an implantable medical device (IMD) comprising: a flexible distal portion extending to a device distal end of the IMD, the flexible distal portion comprising: a first electrode extending distally from the device distal end; and a first fixation element disposed at or around the device distal end; a flexible proximal portion extending to a device proximal end of the IMD, the flexible proximal portion comprising: a second electrode extending proximally from the device proximal end; and a second fixation element disposed at or around the device distal end; a medial portion extending from the flexible distal portion to the flexible proximal portion, the medial portion defining a rigid housing, wherein the first fixation element is configured to be affixed within wall tissue of an atrium of a heart of the patient to cause the first electrode to contact the wall tissue of the atrium, and wherein the second fixation element is configured to be affixed within wall tissue of a ventricle of a heart of the patient to cause the second electrode to contact the wall tissue of the ventricle.

Various examples are described in this disclosure. It is not intended to provide an exclusive or exhaustive explanation of the methods and systems described in detail within the accompanying drawings and description below.

### BRIEF DESCRIPTION OF DRAWINGS

The details of one or more examples of this disclosure are set forth in the accompanying drawings and the description below. Other features, objects, and advantages of this disclosure will be apparent from the description and drawings, and from the claims.

FIG. 1 is a conceptual diagram illustrating an example device implanted in the heart of a patient, in accordance with one or more aspects of this disclosure.

FIG. 2A is a conceptual diagram illustrating an example of the device of FIG. 1.

FIG. 2B is a conceptual diagram illustrating another example of the device of FIG. 1.

FIG. 3 is a block diagram illustrating an example configuration of an example device of any of FIGS. 1–2B.

FIG. 4 is a flowchart illustrating an example process for implanting an example device of any of FIGS. 1–3 within a heart of a patient.

### DETAILED DESCRIPTION

In general, this disclosure is directed to implantable medical devices (IMDs). More particularly, this disclosure is directed to IMDs configured to deliver therapy signals (e.g., defibrillation signals, pacing signals) to a heart of the patient. While the IMD described below is primarily described as being wholly implanted within the vasculature, e.g., the heart, of the patient, the IMD may be at least partially disposed within an epicardial region of the patient or may be implanted within another body lumen of the patient.

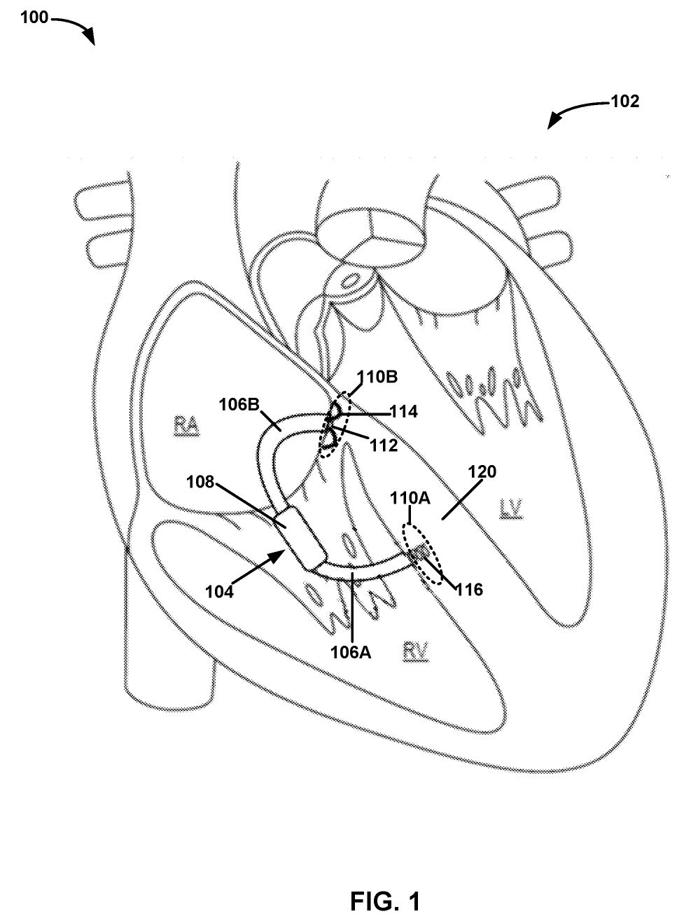

FIG. 1 is a conceptual diagram illustrating an example implantable medical device (IMD) 104 (alternatively referred to herein as “device 104”) implanted in heart 102 of a patient, in accordance with one or more aspects of this disclosure. Device 104 may include a distal portion 106A implanted within a first chamber of heart 102, a proximal portion 106B disposed within second chamber of heart 104, and a medial section 108 disposed between and connecting distal portion 106A to proximal portion 106B.

Distal portion 106A may be implanted within heart 102 to enable an electrode 116 to access a target location 110A within heart 102. In the example illustrated in FIG. 1A, distal section 106A is disposed within a right ventricle (RV) of heart 102 and target location 110A is located along septum 120 of heart 102. In such examples, target location 110A may be disposed at or around a left bundle branch (LBB) of heart 102 in septum 120. In some examples, distal portion 106A may be disposed within another chamber of heart 102 (e.g., right atrium (RA), left atrium (LA), left ventricle (LV)) and target location 110A may be located at another position within heart 102. For example, target location 110A may be located at or around a Triangle of Koch within the RA, a Bundle of His, high septum, LBB, LBB area, or conduction system of the patient.

Distal portion 106A may define a flexible body, e.g., to allow for flexure of distal portion 106A within the first chamber of heart 102. In some examples, as illustrated in FIG. 1, the flexibility of distal portion 106A may allow distal portion 106A to extend from an end of medial portion 108 to wall tissue of the first chamber at or around target location 110A. Distal portion 106A may include one or more distal electrodes 116 disposed at or around a distal end of distal portion 106A. Distal electrode(s) 116 may include, but are not limited to, helical electrodes, button electrodes, or the like. Device 104 may deliver a first electrical stimulation signal to target location 110A via distal electrode(s) 116. The first electrical stimulation signal may include, but are not limited to, cardiac pacing signals (e.g., brady pacing, VF induction pacing, post-shock pacing, and ATP, etc.), cardiac defibrillation shock signals, or the like.

Distal portion 106A may include one or more fixation elements disposed at or around a distal end of distal portion 106A. The one or more fixation elements may engage with wall tissue of heart 102 at or around target location 110A, e.g., to affix distal portion 106A to the wall tissue and place distal electrode(s) 116 in contact with the wall tissue. The one or more fixation elements may include, but are not limited to, fixation tine(s), fixation barb(s), fixation heli(ces), or the like. Fixation element(s) may define distal electrode(s) 116 or may be separate from distal electrode(s) 116.

Proximal portion 106B may be disposed within a second chamber of heart 102. The second chamber of heart 102 may include, but is not limited to, an atrium (e.g., RA) of heart 102. Proximal portion 106B may be affixed to wall tissue of the second chamber of heart 102 at or around target location 110B within the second chamber. Proximal portion 106B may be a flexible body. The flexibility of proximal portion 106B may allow proximal portion 106B to extend from one end of medial portion 108 to target location 110B. Proximal portion 106B may define same or different dimensions than distal portion 106A.

Proximal portion 106B may include one or more proximal electrodes 112 disposed at or around a proximal end of proximal portion 106B. Proximal electrode(s) 112 may deliver a second electrical stimulation signal to target location 110B. Proximal portion 106B may include one or more fixation element(s) 114 at or around the proximal end of proximal portion 106B, e.g., to affix proximal portion 106B to wall tissue of the second chamber of heart 102 at or around target location 110B. Proximal electrode(s) 112 may be of a same or different type as distal electrode(s) 112. Fixation element(s) 114 may be of a same or different type as fixation element(s) on distal portion 106A. For example, as illustrated in FIG. 1, proximal portion 106B may include a button electrode as proximal electrode 112 and fixation tine(s) as fixation element(s) 114 and distal portion 106A may include a helical electrode as both distal electrode 116 and fixation element for distal portion 106A.

Device 104 may include medial portion 108 disposed between and connected to distal portion 106A and proximal portion 106B. Medial portion 108 may define a rigid body. For example, medial portion 108 may include a rigid housing. Medial portion 108 may be connected to distal portion 106A at one end and may be connected to proximal portion 106B at an opposite end.

Medial portion 108 may retain electronic components of device 104. Electronic components may include, but are not limited to, processing circuitry, power source(s), signal generation circuitry, sensing circuitry, or the like. Medial portion 108 may include one or more reference electrodes. Reference electrode(s) may be disposed on an outer surface of a rigid housing of medial portion 108. Reference electrode(s) may provide a common reference electrode for both distal electrode 116 and proximal electrode 112.

When implanted, device 104 may extend between two separate chambers of heart 102, e.g., through a valve connecting the two separate chambers of heart 102. For example, device 104 may extend through a tricuspid valve or a mitral valve of heart 102. Medial portion 108 may be disposed within one chamber of the two separate chambers of heart 102. For example, medial portion 108 may be disposed within RA or RV of heart 102. While FIG. 1 illustrates distal portion 106A being implanted within RV of heart 102 and proximal portion 106B being implanted within RA of heart 102, in some examples, distal portion 106A may be implanted within RA of heart 102 and proximal portion 106B may be implanted within RV of heart 102.

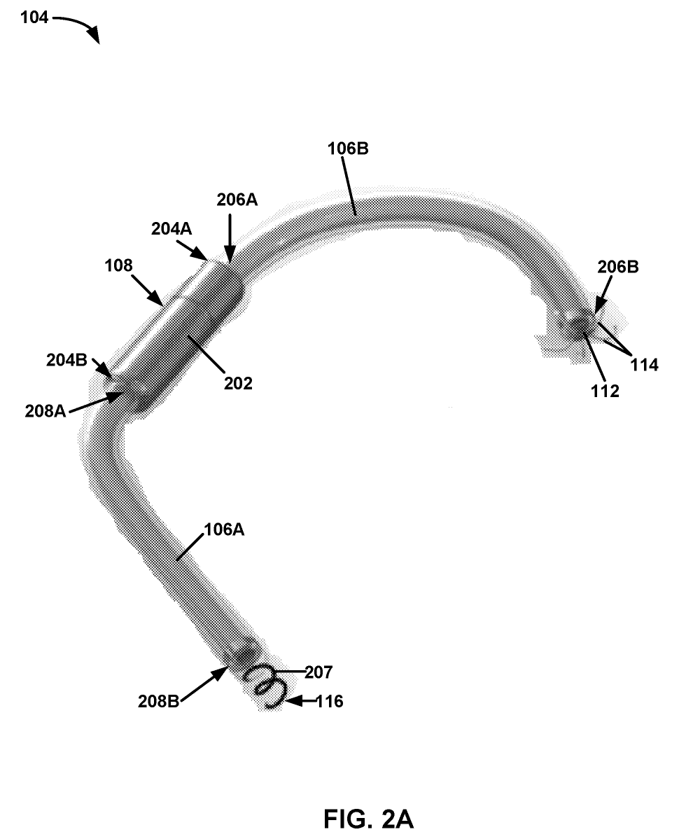

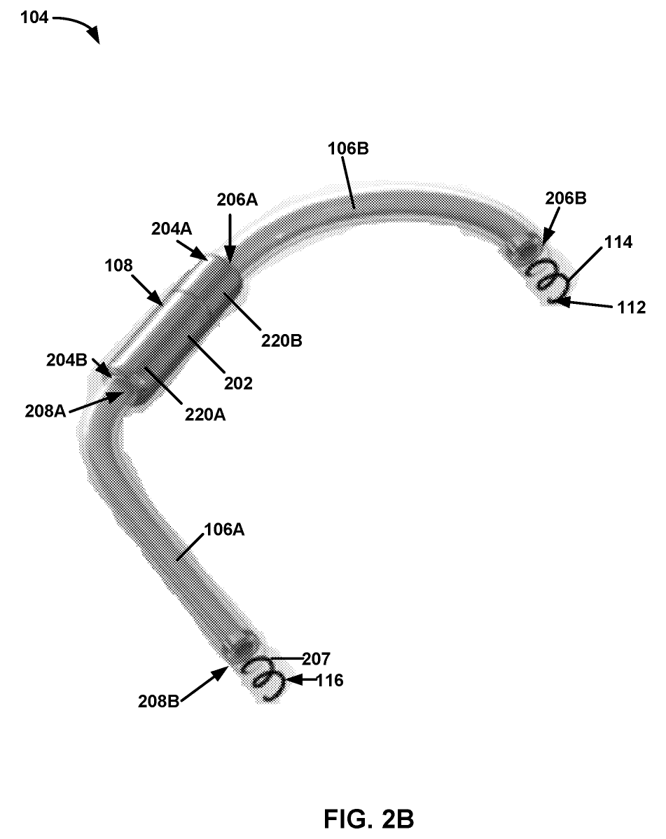

FIG. 2A is a conceptual diagram illustrating an example of device 104 of FIG. 1. FIG. 2B is a conceptual diagram illustrating another example of device 104 of FIG. 1. Device 104 may include one or more combinations of the components illustrated in FIGS. 2A–2B and described herein.

Medial portion 108 of device 104 may define a rigid housing 202. Rigid housing 202 may define a hermetically sealed cavity configured to contain at least some electronic components of device 104. Rigid housing 202 may be formed from a conductive material including titanium or titanium alloy, stainless steel, MP35N (a non-magnetic nickel-cobalt-chromium-molybdenum alloy), platinum alloy or other bio-compatible metal or metal alloy, or other suitable conductive material. In some examples, rigid housing 202 is formed from a non-conductive material including ceramic, glass, sapphire, silicone, polyurethane, epoxy, acetyl co-polymer plastics, polyether ether ketone (PEEK), a liquid crystal polymer, other biocompatible polymer, or other suitable non-conductive material.

Rigid housing 202 may extend from proximal housing end 204A to distal housing end 204B. Rigid housing 202 may define a uniform or a variable outer diameter from proximal housing end 204A to distal housing end 204B. Rigid housing 202 may be cylindrical or substantially cylindrical, but may be other shapes, e.g., prismatic or other geometric shapes. Each of proximal housing end 204A or distal housing end 204B may define a flat surface (e.g., orthogonal to a longitudinal axis of rigid housing 202), a domed surface, an angled surface, or any other shaped surfaces.

All, substantially all, or a portion of rigid housing 202 may function as a reference electrode. The reference electrode may function as an anode electrode during pacing and/or sensing, e.g., while distal electrode 116 and/or proximal electrode 112 functions as the cathode electrode(s). The reference electrode can fully or partially circumscribe rigid housing 202. The reference electrode may extend as a single continuous component or can also include multiple segments spaced a distance apart along a longitudinal axis of and/or around a perimeter of rigid housing 202.

When rigid housing 202 is formed from a conductive material, portions of rigid housing 202 may be electrically insulated by a non-conductive material such as a coating of parylene, polyurethane, silicone, epoxy or other biocompatible polymer, or other suitable material. For the portions of rigid housing 202 without the non-conductive material, one or more discrete areas of rigid housing 202 with conductive material can be exposed to define the reference electrode.

Rigid housing 202 of medial portion 108 may be connected to proximal portion 106B at proximal housing end 204A and may be connected to distal portion 106A at distal housing end 204B. Distal portion 106A may define an elongated body extending from distal portion proximal end 208A to distal portion distal end 208B. Distal portion proximal end 208A of distal portion 106A may be coupled to (e.g., removable coupled to, permanently coupled to) distal housing end 204B of rigid housing 202. Rigid housing may be hermetic and may include hermetic electrical feedthroughs to allow conductors in portions 106 to be electrically connected to circuitry within the hermetic housing.

The elongated body of distal portion 106A may be flexible between proximal end 208A and distal end 208B. Distal portion 106A may be formed from an inner flexible coil and an outer jacket disposed over the inner flexible coil, which may be electrically coupled to distal electrode 116. The inner flexible coil may be formed from a metallic alloy including, but is not limited to, stainless steel. The outer jacket may be formed from a biocompatible polymer including, but are not limited to, polyurethane. The outer jacket may electrically insulate the inner flexible coil and/or components within distal portion 106A from an external environment surrounding distal portion 106A.

Distal portion 106A may include a fixation element 207 and distal electrode 116 at or around distal end 208B. For example, as illustrated in FIG. 2A, distal electrode 116 and fixation element 207 may extend distally from distal end 208B. In some examples, as illustrated in FIGS. 2A and 2B, fixation element 207 may define distal electrode 116, e.g., at or around a distal end of fixation element 207. In some examples, fixation element 207 is separate from distal electrode 116. Fixation element 207 may include, but are not limited to, fixation tine(s), a fixation helix, or the like. Distal electrode 116 may include, but are not limited to, a button electrode or a helical electrode (e.g., as illustrated in FIG. 2A). Fixation element 207 may be configured to penetrate tissue of heart 102 to affix distal end 208B of distal portion 106A at or around target location 110A in heart 102.

Proximal portion 106B may define an elongated body extending from proximal end 206B to distal end 206A. Distal end 206A of proximal portion 106B may be coupled to (e.g., removable coupled to, permanently coupled to) distal housing end 204B of rigid housing 202. The elongated body of proximal portion 106B may be flexible between proximal end 206B and distal end 206A. Proximal portion 106B may be formed from an inner flexible coil, which may be electrically coupled to proximal electrode 112, and an outer jacket disposed over the inner flexible coil. The inner flexible coil may be formed from a metallic alloy including, but is not limited to, stainless steel. The outer jacket may be formed from a biocompatible polymer including, but are not limited to, polyurethane. The outer jacket may electrically insulate the inner flexible coil and/or components within proximal portion 106B from an external environment surrounding proximal portion 106B. Proximal portion 106B may define a same or different dimensions (e.g., length, outer diameter) as distal portion 106A.

In some examples, rigid housing 202 defines a monolithic body. In some examples, rigid housing 202 may be formed by coupling first portion 220A to second portion 220B (e.g., as illustrated in FIG. 2B). First portion 220A may be coupled to distal portion 106A and second portion 220B may be coupled to proximal portion 106B. Different examples of first portion 220A may be coupled to distal portion 106A with different lengths. Different examples of second portion 220B may be coupled to proximal portion 106B with different lengths. Depending on the positions of target locations 110A, 110B within heart 102, a manufacturing assembly may adjustably pair different combinations of first portion 220A and second portion 220B to assembly rigid housing 202, e.g., such that device 104 includes distal portion 106A and proximal portion 106B with corresponding lengths to allow device 104 to reach target locations 110A, 110B. First portion 220A and second portion 220B may allow deice 104 to be modular, e.g., to be implanted to deliver electrical stimulation therapy to a range of possible target locations within heart 102.

Proximal portion 106B may include a fixation element 114 and proximal electrode 112 at or around proximal end 206B. Proximal electrode 112 and fixation element 114 may extend proximally from proximal end 206B. In some examples, as illustrated in FIG. 2B, fixation element 114 may define proximal electrode 112, e.g., at or around an end of fixation element 114. In some examples, as illustrated in FIG. 2A, fixation element 114 is separate from proximal electrode 112. Fixation element 207 may include, but are not limited to, fixation tine(s), a fixation helix, or the like. Distal electrode 116 may include, but are not limited to, a button electrode (e.g., as illustrated in FIG. 2A) or a helical electrode (e.g., as illustrated in FIG. 2B).

Proximal portion 106B may have a same or different fixation element and electrode arrangement as distal portion 106A. For example, as illustrated in FIG. 2A, proximal portion 106B may have a different arrangement for fixation element 114 and proximal electrode 112 (e.g., fixation element 114 being fixation tine(s) and proximal electrode 112 being a button electrode) than the arrangement of fixation element 207 and distal electrode 116 on distal portion 106A (e.g., fixation element 207 being a fixation helix, the fixation helix defining distal electrode 116). In some examples, as illustrated in FIG. 2B, proximal portion 106B may have a same arrangement for fixation element 114 and proximal electrode 112 as the arrangement of fixation element 207 and distal electrode 116 on distal portion 106A. For example, fixation elements 114, 207 may define different fixation helices, with each fixation helix defining a corresponding electrode of electrodes 112, 116. In such examples, fixation elements 114, 207 may define same or different dimensions.

Fixation elements 114, 207 may define fixation tine(s). Fixation tine(s) may be formed from a shape memory material including, but not limited to, Nitinol. Fixation tine(s) may transition between a constrained configuration and an unconstrained configuration (e.g., as illustrated in FIG. 2A). In the unconstrained configuration, Fixation tine(s) may extend radially away from and curve back towards an end of device 104 (e.g., towards proximal end 206B or distal end 208B). Each of the fixation tine(s) may terminate in a distal tip configured to penetrate tissue of the patient. The fixation tine(s) may transition from the constrained configuration to the unconstrained configuration. During the transition, the fixation tine(s) may penetrate cardiac tissue of heart 102 (e.g., at or around target location 110A and/or 110B) and affix distal portion 106A and/or proximal portion 106B to the cardiac tissue.

Each of electrodes 112, 116 may be placed in contact with and/or within cardiac tissue of heart 102. Each of electrodes 112, 116 may be aa button electrode, a spring electrode, a helical electrode, or the like. Each of electrodes 112, 116 may define an electrically active region and may be configured to transmit electrical signals (e.g., cardiac pacing signal, a defibrillation shock electrode) to and/or receive electrical signals from the cardiac tissue in contact with electrodes 112 and/or 116 via the electrically active region. Each of electrodes112, 116 may be formed from an electrically conductive material, such as titanium, platinum, iridium, tantalum, stainless steel, or alloys thereof. For example, distal electrode 116 may be formed from one or more of Platinum Iridium, a Platinum Iridium-clad alloy (e.g., Platinum Iridium-clad Titanium or Nitinol), Nitinol, or Tantalum Tungsten. In some examples, at least a portion of each of electrodes 112, 116 may be coated within an electrically insulating coating, e.g., a parylene, polyurethane, silicone, epoxy, or other insulating coating, to limit an electrically conductive active surface area of each of electrodes 112, 116 and define the respective electrically active regions.

Device 104 may include a delivery tool interface member (not pictured in FIGS. 2A and 2B). The delivery tool interface member may be disposed on or coupled to rigid housing 202 and/or to proximal portion 106B. The delivery tool interface member may engage with a delivery tool (e.g., a tether assembly) operated by the clinician. The clinician may navigate device 104 within the vasculature of the patient by applying forces (e.g., pushing, pulling, rotary forces) on the delivery tool interface member via the delivery tool. The delivery tool interface member may be electrically conductive and may transmit electrical signals (e.g., from electrical components of device 104) along the delivery tool to an external computing device operated by the clinician.

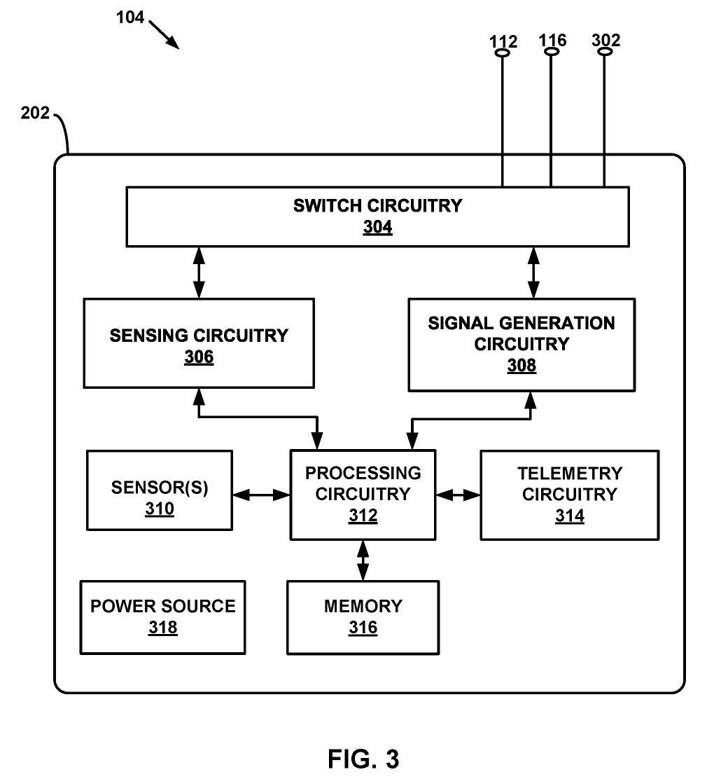

FIG. 3 is a block diagram illustrating an example configuration of an example device 104 of any of FIGS. 1–2B. As illustrated in FIG. 3, device 104 include distal electrode 116, proximal electrode 112, and reference electrode 302 (e.g., disposed on rigid housing 202), each of which may be configured as described with respect to FIGS. 1–2B. In the example shown in FIG. 3, device 104 includes switch circuitry 304, sensing circuitry 306, signal generation circuitry 308, sensor(s) 310, processing circuitry 312, telemetry circuitry 314, memory 316, and power source 318. The various circuitry may be, or include, programmable or fixed function circuitry configured to perform the functions attributed to respective circuitry. Memory 316 may store computer-readable instructions that, when executed by processing circuitry 312, cause device 104 to perform various functions. Memory 316 may be a storage device or other non-transitory medium. The components of device 104 illustrated in FIG. 3 may be housed within rigid housing 202. In some examples, the components of device 104 illustrated in FIG. 3 may be connected (e.g., to electrodes 112, 116,) via conductor(s) disposed within and extending along distal portion 106A and/or proximal portion 106B.

Signal generation circuitry 308 generates electrical therapy signals, e.g., cardiac pacing signals, defibrillation signals. Switch circuitry 304 is coupled to electrodes 112, 116, and 302 and may include one or more switch arrays, one or more multiplexers, one or more switches (e.g., a switch matrix or other collection of switches), one or more transistors, or other electrical circuitry. Switch circuitry 304 is configured to direct therapy signals from signal generation circuitry 308 to a selected combination of electrodes 112, 116, and 302, having selected polarities, e.g., to selectively deliver pacing pulses to the RA, ventricles, or interventricular septum of heart 102 and/or to deliver defibrillation shock signals to heart 102. Device 104 may deliver the therapy signals to different chambers of heart 102 simultaneously, sequentially, or in any other combination. In some examples, processing circuitry 312 may control switch circuitry 304, sensing circuitry 306, and signal generation circuitry 308 to sense and pace in an atrium and ventricle to provide atrioventricular synchronous pacing. Switch circuitry 304 may couple distal electrode 116 and proximal electrode 112 to signal generation circuitry 308 as cathode, and reference electrode 302 to signal generation circuitry 308 as an anode.

Switch circuitry 304 may also selectively couple sensing circuitry 306 to selected combinations of electrodes (e.g., distal electrode 116, proximal electrode 112, and/or reference electrode 302, e.g., to selectively sense the electrical activity of one or more chambers of heart 102. Sensing circuitry 306 may include filters, amplifiers, analog-to-digital converters, or other circuitry configured to sense cardiac electrical signals via the selected electrodes. For example, switch circuitry 304 may couple different electrodes to respective sensing channels provided by sensing circuitry 306 to respectively sense either ventricular or atrial cardiac electrical signals. In some examples, sensing circuitry 306 is configured to detect events, e.g., depolarizations, within the cardiac electrical signals, and provide indications thereof to processing circuitry 312. Processing circuitry 312 may control the delivery of electrical stimulation to target locations 110A and/o 110B based at least in part on the sensed cardiac electrical signals. Processing circuitry 312 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 312 herein may be embodied as firmware, hardware, software or any combination thereof.

Sensor(s) 310 may include one or more sensing elements that transduce patient physiological activity to an electrical signal to sense values of a respective patient parameter, e.g., electrical stability of one or more chambers of heart 102 (e.g., ventricular electrical stability of heart 102) and/or a hemodynamic status of the patient. Sensor(s) 310 may include one or more accelerometers, optical sensors, chemical sensors, temperature sensors, pressure sensors, strain gauges, other mechanical sensors, or any other types of sensors. Sensor(s) 310 may output patient parameter values that may be used as feedback to control sensing and delivery of therapy by device 104.

Telemetry circuitry 314 supports wireless communication between device 104 and an external programmer (not shown in FIG. 3) or another computing device under the control of processing circuitry 312. Processing circuitry 312 of device 104 may receive, as updates to operational parameters from the computing device, and provide collected data, e.g., sensed heart activity or other patient parameters, via telemetry circuitry 314. Telemetry circuitry 314 may accomplish communication by radiofrequency (RF) communication techniques, e.g., via an antenna (not shown).

Power source(s) 318 delivers operating power to various components of device 104. Power source(s) 318 may include a rechargeable or non-rechargeable battery and a power generation circuit to produce the operating power. Recharging may be accomplished through proximal inductive interaction between an external charger and an inductive charging coil within device 104. In some examples, device 104 includes two or more power sources 318.

In some examples, device 104 may include one or more power generation components (not pictured in FIG. 3) coupled to power sources 318. The one or more power generation components may be disposed within one or more of distal portion 106A, medial section 108, or proximal portion 106B. The one or more power generation components may convert forces applied on device 104 (e.g., resulting from mechanical movement of heart 102) to electrical power and may store the generated electrical power within power sources 318.

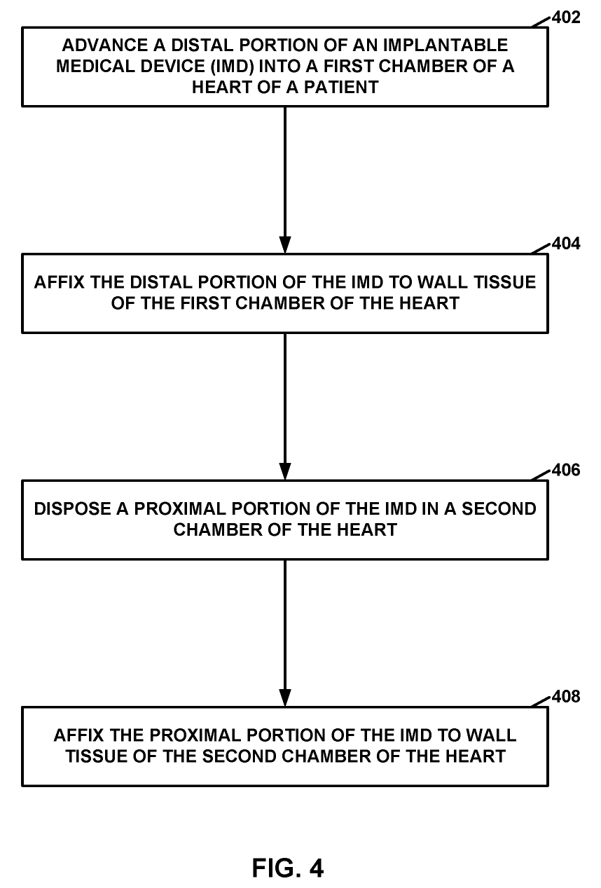

FIG. 4 is a flowchart illustrating an example process for implanting an example device of any of FIGS. 1–3 within a heart of a patient. While the technique illustrated in FIG. 4 is primarily described with respect to device 104, the technique may be performed using any other example device described herein.

A clinician may advance a distal portion 106A of an implantable medical device (IMD) 104A into a chamber of heart 102 of a patient (402). Device 104 may include a first elongated body defining distal portion 106A, a second elongated body defining proximal portion 106B, and medial portion 108 connecting distal portion 106A to proximal portion 106B. Distal portion 106A and proximal portion 106B may be flexible and medial portion 108 may be rigid, e.g., may define a rigid housing 202. Device 104 may be coupled to a tether assembly via a delivery tool interface member disposed on medial portion 108 or proximal portion 106B of device 104. Device 104 may be disposed within a delivery catheter. Distal portion may be positioned distal to medial portion 108 and proximal portion 106B within the delivery catheter. The delivery catheter may constrain fixation elements on device 104 (e.g., fixation element 114, 207) into a constrained configuration.

The clinician may advance a distal portion of the delivery catheter (e.g., containing distal portion 106A of device 104) from an incision location, through the vasculature of the patient, and into a first chamber of heart 102. The first chamber may be an atrium or a ventricle (e.g., the RV) of heart 102. The clinician may advance the delivery catheter through a second chamber of heart 102 and into the first chamber of heart 102 (e.g., via a valve connecting the first chamber to the second chamber). Within the first chamber, the clinician may position a distal opening of the delivery catheter at or around target location 110A within the first chamber.

The clinician may affix distal portion 106A of IMD 104 to wall tissue of the chamber of heart 102 (404). The clinician may advance distal portion 106A out of the delivery catheter to affix distal portion 106A to the wall tissue, e.g., at or around target location 110A. In some examples, when fixation element(s) 207 (e.g., fixation tine(s)) on distal portion 106A are unconstrained by the delivery catheter, fixation element(s) 207 may expand towards the unconstrained configuration and penetrate the wall tissue, thereby affixing distal portion 106A to the wall tissue. In some examples, the clinician causes fixation element(s) 207 (e.g., a fixation helix) on distal portion 106A to penetrate the wall tissue, e.g., by rotating distal portion 106A about a longitudinal axis of distal portion 106A using a handle of the tether assembly.

When distal portion 106A is affixed to the wall tissue, distal electrode 116 on distal portion 106A may be placed in contact with a surface of the wall tissue at target location 110A and/or may be disposed within the wall tissue at target location 110A. Device 104 may deliver electrical stimulation signals (e.g., cardiac pacing signals, defibrillation shock signals) to and/or sense electrical signals from the wall tissue using distal electrode 116.

The clinician may dispose proximal portion 106B of IMD 104 into a second chamber of heart 102 (406). The clinician may position medial portion 108 of IMD 104 in the first chamber or the second chamber of heart 102. At least a portion of proximal portion 106B may be disposed within the second chamber and a remainder of device 104 may extend into the first chamber through a valve (e.g., tricuspid valve, mitral valve) connecting the first chamber to the second chamber. The clinician may further retract the delivery catheter relative to device 104 to expose medial section 108 and proximal portion 106B. The clinician may position orient, via the tether assembly, proximal portion 106B towards target location 110B in the second chamber.

The clinician may affix proximal portion 106B of IMD 104 to wall tissue of the second chamber of heart 102 (408). In some examples, the clinician may cause, via the tether assembly, fixation element(s) 114 (e.g., fixation tine(s)) on proximal portion 106B to expand towards the unconstrained configuration and penetrate the wall tissue, thereby affixing proximal portion 106B to the wall tissue. In some examples, the clinician causes fixation element(s) 114 (e.g., a fixation helix) on proximal portion 106B to penetrate the wall tissue, e.g., by rotating proximal portion 106B about a longitudinal axis of proximal portion 106B using a handle of the tether assembly.

When proximal portion 106B is affixed to the wall tissue, proximal electrode 112 on proximal portion 106B may be placed in contact with a surface of the wall tissue at target location 110B and/or may be disposed within the wall tissue at target location 110B. Device 104 may deliver electrical stimulation signals (e.g., cardiac pacing signals, defibrillation shock signals) to and/or sense electrical signals from the wall tissue using proximal electrode 112.

It may be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

In addition, it should be noted that system described herein may not be limited to treatment of a human patient. In alternative examples, the system may be implemented in non-human patients, e.g., primates, canines, equines, pigs, and felines. These other animals may undergo clinical or research therapies that may benefit from the subject matter of this disclosure.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

This disclosure describes each of the following examples.

Example 1: an implantable medical device (IMD) comprising: a flexible distal portion extending to a device distal end of the IMD, the flexible distal portion comprising: a first electrode extending distally from the device distal end; a flexible proximal portion extending to a device proximal end of the IMD, the flexible proximal portion comprising: a second electrode extending proximally from the device proximal end; and a medial portion extending from the flexible distal portion to the flexible proximal portion, the medial portion defining a rigid housing, wherein the flexible distal portion is configured to be at least partially disposed within a first chamber of a heart of a patient to cause the first electrode to contact wall tissue of the first chamber of the heart, and wherein the flexible proximal portion is configured to be at least partially disposed within a second chamber of the heart to cause the second electrode to contact wall tissue of the second chamber of the heart.

Example 2: the IMD of example 1, further comprising: a first fixation element disposed at or around the device distal end, the first fixation element being configured to affix the flexible distal portion of the IMD to the wall tissue of the first chamber of the heart; and a second fixation element disposed at or around the device proximal end, the second fixation element being configured to affix the flexible proximal portion of the IMD to the wall tissue of the second chamber of the heart.

Example 3: the IMD of example 2, wherein each of the first fixation element or the second fixation element comprises one or more of: one or more fixation tines; or a fixation helix.

Example 4: the IMD of any of examples 2 and 3, wherein one or more of: at least a first portion of the first fixation element defines the first electrode; or at least a second portion of the second fixation element defines the second electrode.

Example 5: the IMD of any of examples 1–4, wherein the rigid housing is configured to retain one or more of: a power source of the IMD; processing circuitry of the IMD; or signal generation circuitry electrically coupled to at least one of the first electrode or the second electrode.

Example 6: the IMD of any of examples 1–5, wherein the rigid housing defines a third electrode extending at least partially around an outer surface of the rigid housing.

Example 7: the IMD of example 6, wherein the third electrode defines a return electrode for one or more of the first electrode or the second electrode.

Example 8: the IMD of any of examples 1–7, wherein the IMD is configured to: deliver a first electrical signal to the wall tissue of the first chamber via the first electrode; and deliver a second electrical signal to the wall tissue of the second chamber via the second electrode.

Example 9: the IMD of example 8, wherein the IMD is configured to deliver the first electrical signal to the wall tissue of the first chamber at or around a left bundle branch (LBB) of the patient.

Example 10: the IMD of any of examples 1–9, wherein one or more of the flexible distal portion or the flexible proximal portion comprises an elongated body, the elongated body comprising: a flexible coil along a longitudinal length of the elongated body, the flexible coil being formed from a metallic alloy, one or more electrical connectors extending along the longitudinal length of the elongated body; and a biocompatible polymer disposed along the longitudinal length of the elongated body and radially outwards of the flexible coil and the one or more electrical connectors.

Example 11: the IMD of any of examples 1–10, wherein the flexible distal portion or the flexible proximal portion is configured to extend through a valve between the first chamber and the second chamber.

Example 12: the IMD of any of examples 1–11, wherein the first chamber comprises a ventricle of the heart of the patient, and wherein the second chamber comprises an atrium of the heart of the patient.

Example 13: a method comprising: advancing an implantable medical device (IMD) within vasculature of a patient into a heart of the patient, the IMD comprising: a flexible distal portion extending to a device distal end of the IMD, the flexible distal portion comprising: a first electrode extending distally from the device distal end, a flexible proximal portion extending to a device proximal end of the IMD, the flexible proximal portion comprising: a second electrode extending proximally from the device proximal end, and a medial portion extending from the flexible distal portion to the flexible proximal portion, the medial portion defining a rigid housing; advancing at least a portion of the flexible distal portion into a first chamber of the heart; affixing the flexible distal portion to wall tissue of the first chamber to cause the first electrode to contact the wall tissue of the first chamber; disposing at least a portion of the flexible proximal portion in a second chamber of the heart; affixing the flexible proximal portion to wall tissue of the second chamber to cause the second electrode to contact the wall tissue of the second chamber; delivering, by the IMD and via the first electrode, a first electrical signal to the wall tissue of the first chamber; and delivering, by the IMD and via the second electrode, a second electrical to the wall tissue of the second chamber.

Example 14: the method of example 13, wherein the IMD further comprises: a first fixation element disposed at or around the device distal end; and a second fixation element disposed at or around the device proximal end, wherein affixing the flexible distal portion to the wall tissue of the first chamber comprises affixing the first fixation element to the wall tissue of the first chamber; and wherein affixing the flexible proximal portion to the wall tissue of the second chamber comprises affixing the second fixation element to the wall tissue of the second chamber.

Example 15: the method of example 14, wherein each of the first fixation element or the second fixation element comprises one or more of: one or more fixation tines; or a fixation helix.

Example 16: the method of any of examples 14 and 15, wherein one or more of: at least a first portion of the first fixation element defines the first electrode; or at least a second portion of the second fixation element defines the second electrode.

Example 17: the method of any of examples 13–16, wherein the rigid housing is configured to retain one or more of: a power source of the IMD; processing circuitry of the IMD; or signal generation circuitry electrically coupled to at least one of the first electrode or the second electrode.

Example 18: the method of any of examples 13–17, wherein the rigid housing defines a third electrode extending at least partially around an outer surface of the rigid housing.

Example 19: the method of example 18, wherein the third electrode defines a return electrode for one or more of the first electrode or the second electrode.

Example 20: the method of any of examples 13–19, wherein one or more of the flexible distal portion or the flexible proximal portion comprises an elongated body, the elongated body comprising: a flexible coil along a longitudinal length of the elongated body, the flexible coil being formed from a metallic alloy, one or more electrical connectors extending along the longitudinal length of the elongated body; and a biocompatible polymer disposed along the longitudinal length of the elongated body and radially outwards of the flexible coil and the one or more electrical connectors.

Example 21: the method of any of examples 13–20, wherein the first chamber comprises a ventricle of the heart of the patient, and wherein the second chamber comprises an atrium of the heart of the patient.

Example 22: an implantable medical device (IMD) comprising: a flexible distal portion extending to a device distal end of the IMD, the flexible distal portion comprising: a first electrode extending distally from the device distal end; and a first fixation element disposed at or around the device distal end; a flexible proximal portion extending to a device proximal end of the IMD, the flexible proximal portion comprising: a second electrode extending proximally from the device proximal end; and a second fixation element disposed at or around the device distal end; a medial portion extending from the flexible distal portion to the flexible proximal portion, the medial portion defining a rigid housing, wherein the first fixation element is configured to be affixed within wall tissue of an atrium of a heart of the patient to cause the first electrode to contact the wall tissue of the atrium, and wherein the second fixation element is configured to be affixed within wall tissue of a ventricle of a heart of the patient to cause the second electrode to contact the wall tissue of the ventricle.

Example 23: the IMD of example 22, wherein one or more of: the first fixation element defines the first electrode; or the second fixation element defines the second electrode.

Example 24: the IMD of claim 22, wherein one or more of: the first electrode is separate from the first fixation element; or the second electrode is separate from the second fixation element.

Example 25: the IMD of any of examples 22–24, wherein each of the first fixation element or the second fixation element comprises one or more of: one or more fixation tines; or a fixation helix.

Example 26: the IMD of any of examples 22–25, wherein the rigid housing is configured to retain one or more of: a power source of the IMD; processing circuitry of the IMD; or signal generation circuitry electrically coupled to at least one of the first electrode and the second electrode.

Example 27: the IMD of any of examples 22–26, wherein the rigid housing defines a third electrode extending at least partially around an outer surface of the rigid housing.

Example 28: the IMD of example 27, wherein the third electrode defines a return electrode for one or more of the first electrode or the second electrode.

Example 29: the IMD of any of examples 22–28, wherein the IMD is configured to: deliver a first electrical signal to the wall tissue of the atrium via the first electrode; and deliver a second electrical signal to the wall tissue of the ventricle via the second electrode.

Example 30: the IMD of example 29, wherein the IMD is configured to deliver the first electrical signal to the wall tissue of the first chamber at or around a left bundle branch (LBB) of the patient.

Example 31: the IMD of any of examples 22–30, wherein one or more of the flexible distal portion or the flexible proximal portion comprises an elongated body, the elongated body comprising: a flexible coil along a longitudinal length of the elongated body, the flexible coil being formed from a metallic alloy, one or more electrical connectors extending along the longitudinal length of the elongated body; and a biocompatible polymer disposed along the longitudinal length of the elongated body and radially outwards of the flexible coil and the one or more electrical connectors.

Example 32: the IMD of any of examples 22–31, wherein the flexible distal portion or the flexible proximal portion is configured to extend through a tricuspid valve between the atrium and the ventricle.

Various examples have been described. These and other examples are within the scope of the following claims.

What is claimed is:

1. An implantable medical device (IMD) comprising:
a flexible distal portion extending to a device distal end of the IMD, the flexible distal portion comprising:
a first electrode extending distally from the device distal end;
a flexible proximal portion extending to a device proximal end of the IMD, the flexible proximal portion comprising:
a second electrode extending proximally from the device proximal end; and
a medial portion extending from the flexible distal portion to the flexible proximal portion, the medial portion defining a rigid housing,
wherein the flexible distal portion is configured to be at least partially disposed within a first chamber of a heart of a patient to cause the first electrode to contact wall tissue of the first chamber of the heart, and wherein the flexible proximal portion is configured to be at least partially disposed within a second chamber of the heart to cause the second electrode to contact wall tissue of the second chamber of the heart.

2. The IMD of claim 1, further comprising:
a first fixation element disposed at or around the device distal end, the first fixation element being configured to affix the flexible distal portion of the IMD to the wall tissue of the first chamber of the heart; and
a second fixation element disposed at or around the device proximal end, the second fixation element being configured to affix the flexible proximal portion of the IMD to the wall tissue of the second chamber of the heart.

3. The IMD of claim 2, wherein each of the first fixation element or the second fixation element comprises one or more of:
one or more fixation tines; or
a fixation helix.

4. The IMD of claim 2, wherein one or more of:
at least a first portion of the first fixation element defines the first electrode; or
at least a second portion of the second fixation element defines the second electrode.

5. The IMD of claim 1, wherein the rigid housing is configured to retain one or more of:
a power source of the IMD;
processing circuitry of the IMD; or
signal generation circuitry electrically coupled to at least one of the first electrode or the second electrode.

6. The IMD of claim 1, wherein the rigid housing defines a third electrode extending at least partially around an outer surface of the rigid housing.

7. The IMD of claim 1, wherein the IMD is configured to:
deliver a first electrical signal to the wall tissue of the first chamber via the first electrode; and
deliver a second electrical signal to the wall tissue of the second chamber via the second electrode.

8. The IMD of claim 1, wherein one or more of the flexible distal portion or the flexible proximal portion comprises an elongated body, the elongated body comprising:
a flexible coil along a longitudinal length of the elongated body, the flexible coil being formed from a metallic alloy,
one or more electrical connectors extending along the longitudinal length of the elongated body; and
a biocompatible polymer disposed along the longitudinal length of the elongated body and radially outwards of the flexible coil and the one or more electrical connectors.

9. The IMD of claim 1, wherein the flexible distal portion or the flexible proximal portion is configured to extend through a valve between the first chamber and the second chamber.

10. The IMD of claim 1, wherein the first chamber comprises a ventricle of the heart of the patient, and wherein the second chamber comprises an atrium of the heart of the patient.

11. A method comprising:
advancing an implantable medical device (IMD) within vasculature of a patient into a heart of the patient, the IMD comprising:
a flexible distal portion extending to a device distal end of the IMD, the flexible distal portion comprising:
a first electrode extending distally from the device distal end,
a flexible proximal portion extending to a device proximal end of the IMD, the flexible proximal portion comprising:
a second electrode extending proximally from the device proximal end, and
a medial portion extending from the flexible distal portion to the flexible proximal portion, the medial portion defining a rigid housing;
advancing at least a portion of the flexible distal portion into a first chamber of the heart;
affixing the flexible distal portion to wall tissue of the first chamber to cause the first electrode to contact the wall tissue of the first chamber;
disposing at least a portion of the flexible proximal portion in a second chamber of the heart;
affixing the flexible proximal portion to wall tissue of the second chamber to cause the second electrode to contact the wall tissue of the second chamber;
delivering, by the IMD and via the first electrode, a first electrical signal to the wall tissue of the first chamber; and
delivering, by the IMD and via the second electrode, a second electrical to the wall tissue of the second chamber.

12. The method of claim 11, wherein the IMD further comprises:
a first fixation element disposed at or around the device distal end; and
a second fixation element disposed at or around the device proximal end,
wherein affixing the flexible distal portion to the wall tissue of the first chamber comprises affixing the first fixation element to the wall tissue of the first chamber; and
wherein affixing the flexible proximal portion to the wall tissue of the second chamber comprises affixing the second fixation element to the wall tissue of the second chamber.

13. The method of claim 12, wherein each of the first fixation element or the second fixation element comprises one or more of:
one or more fixation tines; or
a fixation helix.

14. The method of claim 12, wherein one or more of:
at least a first portion of the first fixation element defines the first electrode; or
at least a second portion of the second fixation element defines the second electrode.

15. The method of claim 11, wherein the rigid housing is configured to retain one or more of:
a power source of the IMD;
processing circuitry of the IMD; or
signal generation circuitry electrically coupled to at least one of the first electrode or the second electrode.

16. The method of claim 11, wherein one or more of the flexible distal portion or the flexible proximal portion comprises an elongated body, the elongated body comprising:
a flexible coil along a longitudinal length of the elongated body, the flexible coil being formed from a metallic alloy,
one or more electrical connectors extending along the longitudinal length of the elongated body; and
a biocompatible polymer disposed along the longitudinal length of the elongated body and radially outwards of the flexible coil and the one or more electrical connectors.

17. An implantable medical device (IMD) comprising:
a flexible distal portion extending to a device distal end of the IMD, the flexible distal portion comprising:
a first electrode extending distally from the device distal end; and
a first fixation element disposed at or around the device distal end;
a flexible proximal portion extending to a device proximal end of the IMD, the flexible proximal portion comprising:
a second electrode extending proximally from the device proximal end; and
a second fixation element disposed at or around the device distal end;
a medial portion extending from the flexible distal portion to the flexible proximal portion, the medial portion defining a rigid housing,
wherein the first fixation element is configured to be affixed within wall tissue of an atrium of a heart of the patient to cause the first electrode to contact the wall tissue of the atrium, and wherein the second fixation element is configured to be affixed within wall tissue of a ventricle of a heart of the patient to cause the second electrode to contact the wall tissue of the ventricle.

18. The IMD of claim 17, wherein each of the first fixation element or the second fixation element comprises one or more of:
one or more fixation tines; or
a fixation helix.

19. The IMD of claim 17, wherein the rigid housing is configured to retain one or more of:
a power source of the IMD;
processing circuitry of the IMD; or
signal generation circuitry electrically coupled to at least one of the first electrode and the second electrode.

20. The IMD of claim 17, wherein one or more of the flexible distal portion or the flexible proximal portion comprises an elongated body, the elongated body comprising:
a flexible coil along a longitudinal length of the elongated body, the flexible coil being formed from a metallic alloy,
one or more electrical connectors extending along the longitudinal length of the elongated body; and
a biocompatible polymer disposed along the longitudinal length of the elongated body and radially outwards of the flexible coil and the one or more electrical connectors.
